(19) United States
(12) Patent Application Publication
(10) Pub. No.: US 2013/0079774 A1
(43) Pub. Date: Mar. 28, 2013

(54) End-Effector Assemblies for Electrosurgical Instruments and Methods of Manufacturing Jaw Assembly Components of End-Effector Assemblies

(71) Applicants: William Ross Whitney, Boulder, CO (US); Michael B. Lyons, Boulder, CO (US)

(72) Inventors: William Ross Whitney, Boulder, CO (US); Michael B. Lyons, Boulder, CO (US)

(73) Assignee: TYCO Healthcare Group LP, Boulder, CO (US)

(21) Appl. No.: 13/243,628

(22) Filed: Sep. 23, 2011

(51) Int. Cl. A61B 18/14; B23P 11/00; A61B 17/28

(52) U.S. Cl. 606/52; 606/208; 29/428

(57) ABSTRACT

An end-effector assembly includes opposing first and second jaw assemblies pivotably mounted with respect to one another. The first jaw assembly includes a first jaw member including a first arm member defining one or more apertures at least partially therethrough and a first support base extending distally therefrom, wherein an engagement structure of the first arm member is joined to an engagement structure of the first support base. The second jaw assembly includes a second jaw member including a second arm member defining one or more apertures at least partially therethrough and a second support base extending distally therefrom, wherein an engagement structure of the second arm member is joined to an engagement structure of the second support base. One or more pivot pins are engaged with the one or more apertures of the first and second jaw members.

### BACKGROUND

1. Technical Field

The present disclosure relates to electrosurgical instruments. More particularly, the present disclosure relates to end-effector assemblies for use in electrosurgical instruments and methods of manufacturing jaw assembly components of end-effector assemblies.

2. Discussion of Related Art

Electrosurgical instruments have become widely used by surgeons. Electrosurgery involves the application of thermal and/or electrical energy to cut, dissect, ablate, coagulate, cauterize, seal or otherwise treat biological tissue during a surgical procedure. Electrosurgery is typically performed using an electrosurgical generator operable to output energy and a handpiece including a surgical instrument (e.g., end effector) adapted to transmit energy to a tissue site during electrosurgical procedures. A variety of types of end-effector assemblies have been employed for various types of electrosurgery using a variety of types of monopolar and bipolar electrosurgical instruments.

The basic purpose of both monopolar and bipolar electrosurgery is to produce heat to achieve the desired tissue/clinical effect. In monopolar electrosurgery, devices use an instrument with a single, active electrode to deliver energy from an electrosurgical generator to tissue, and a patient return electrode or pad that is attached externally to the patient (e.g., a plate positioned on the patient's thigh or back) as the means to complete the electrical circuit between the electrosurgical generator and the patient. When the electrosurgical energy is applied, the energy travels from the active electrode, to the surgical site, through the patient and to the return electrode. In bipolar electrosurgery, both the active electrode and return electrode functions are performed at the site of surgery, Bipolar electrosurgical devices include two electrodes that are located in proximity to one another for the application of current between their surfaces. Bipolar electrosurgical current travels from one electrode, through the intervening tissue to the other electrode to complete the electrical circuit. Bipolar instruments generally include end-effectors, such as grippers, cutters, forceps, dissectors and the like.

Forceps utilize mechanical action to constrict, grasp, dissect and/or clamp tissue. By utilizing an electrosurgical forceps, a surgeon can utilize both mechanical clamping action and electrosurgical energy to effect hemostasis by heating the tissue and blood vessels to cauterize, coagulate/desiccate, seal and/or divide tissue. Bipolar electrosurgical forceps utilize two generally opposing electrodes that are operably associated with the inner opposing surfaces of end effectors and that are both electrically coupled to an electrosurgical generator. In bipolar forceps, the end-effector assembly generally includes opposing jaw assemblies pivotably mounted with respect to one another. In bipolar configuration, only the tissue grasped between the jaw assemblies is included in the electrical circuit. Because the return function is performed by one jaw assembly of the forceps, no patient return electrode is needed.

By utilizing an electrosurgical forceps, a surgeon can cauterize, coagulate/desiccate and/or seal tissue and/or simply reduce or slow bleeding by controlling the intensity, frequency and duration of the electrosurgical energy applied through the jaw assemblies to the tissue. During the sealing process, mechanical factors such as the pressure applied between opposing jaw assemblies and the gap distance between the electrically-conductive tissue-contacting surfaces (electrodes) of the jaw assemblies play a role in determining the resulting thickness of the sealed tissue and effectiveness of the seal.

Jaw assemblies for use in electrosurgical instruments are required to meet specific tolerance requirements for proper jaw alignment and other closely-toleranced features, and are generally manufactured by expensive and time-consuming processes. Gap tolerances and/or surface parallelism and flatness tolerances are parameters that, if properly controlled, can contribute to a consistent and effective tissue seal. Manufacturing closely-toleranced jaw assemblies typically involves complex machining operations, such as machining of a part from a single piece of material stock or workpiece, or other complex manufacturing processes, such as metal injection molding followed by finishing processes to remove certain injection-molding features such as gate marks, ejector pin marks or parting lines.

### SUMMARY

A continuing need exists for tightly-toleranced jaw assembly components that can be readily integrated into manufacturing assembly processes for the production of end-effector assemblies for use in electrosurgical instruments, such as electrosurgical forceps. Further need exists for the development of a manufacturing process that effectively fabricates jaw assembly components at low cost, and results in the formation of a reliable electrosurgical instrument that meets specific tolerance requirements for proper jaw alignment and other tightly-toleranced jaw assembly features, with reduction or elimination of complex machining operations.

A continuing need exists for a reliable electrosurgical instrument that regulates the gap distance between opposing jaw assemblies, reduces the chances of short circuiting the opposing jaws during activation, and assists in gripping, manipulating and holding tissue prior to and during activation and dividing of the tissue. Further need exists for the development of a manufacturing process that effectively fabricates electrically non-conductive stop members associated with one or both of the opposing jaw assemblies.

According to an aspect, an end-effector assembly is provided. The end-effector assembly includes opposing first and second jaw assemblies pivotably mounted with respect to one another. The first jaw assembly includes a first jaw member including a first arm member defining one or more apertures at least partially therethrough and a first support base extending distally from the first arm member, wherein an engagement structure of the first arm member is joined to an engagement structure of the first support base to thereby form the first jaw member. The second jaw assembly includes a second jaw member including a second arm member defining one or more apertures at least partially therethrough and a second support base extending distally from the second arm member, wherein an engagement structure of the second arm member is joined to an engagement structure of the second support base to thereby form the second jaw member. One or more pivot pins are engaged with the one or more apertures of the first and second jaw members such that the first and second jaw assemblies are pivotably mounted with respect to one another.

According to another aspect, a method of manufacturing an end-effector assembly is provided. The method includes the initial steps of providing a first arm member and a first support base, each including engagement structures configured for attachment to one another, and providing a second arm member and a second support base, each including engagement structures configured for attachment to one another. The first arm member includes one or more pivot holes defined at least partially therethrough. The second arm member includes one or more pivot holes defined at least partially therethrough. The method also includes the steps of joining the engagement structure of the first arm member to the engagement structure of the first support base, joining the engagement structure of the second arm member to the engagement structure of the second support base, and pinning the first and second arm members using the one or more pivot holes of the first and second arm members such that the first and second arm members are pivotably mounted with respect to one another.

In any of the aspects, the first arm member and/or the second arm member may be formed using a fineblanking process. In addition or alternatively, the first support base and/or the second support base may be formed using a fineblanking process.

In any of the aspects, the end-effector assembly may include an insulator adapted to support an electrically-conductive tissue-engaging surface associated with the first jaw assembly and/or the second jaw assembly. The first support base may be configured to support the insulator associated with the first jaw assembly. In addition or alternatively, the second support base may be configured to support the insulator associated with the second jaw assembly.

According to another aspect, a method of manufacturing a jaw member is provided. The method includes the steps of fineblanking a first arm member including a first engagement structure, fineblanking a first support base including a second engagement structure configured to engage with the first engagement structure, and joining the first arm member to the first support base via the first and second engagement structures.

In any of the aspects, the end-effector assembly may include one or more electrically non-conductive stop members disposed on the inner-facing surface of the first jaw assembly and/or the second jaw assembly (or the first support base and/or second support base). The non-conductive stop member(s) may be configured to control the gap distance between the opposing jaw assemblies (and/or jaw members) when tissue is held therebetween, e.g., when the first and second jaw assemblies are in a closed position. The stop members may be disposed on one or both jaw assemblies on opposite sides of a longitudinally-oriented knife channel and/or in an alternating, laterally-offset manner relative to one another along the length of the surface of one or both the jaw assemblies, or portion thereof.

In any of the aspects, one or more non-conductive stop members associated with the inner-facing surface of the first jaw assembly and/or the inner-facing surface of the second jaw assembly (or the first support base and/or second support base) may be formed using a direct write process. A direct write process, e.g., MICROPENNING®, may be used to deposit a dielectric ink on the inner-facing surface of an electrically-conductive tissue-engaging surface associated with the first support base and/or the inner-facing surface of an electrically-conductive tissue-engaging surface associated with second support base.

### BRIEF DESCRIPTION OF THE DRAWINGS

Objects and features of the presently-disclosed end-effector assemblies for use in electrosurgical instruments and methods of manufacturing jaw assembly components of end-effector assemblies will become apparent to those of ordinary skill in the art when descriptions of various embodiments thereof are read with reference to the accompanying drawings, of which:

FIG. 1 is a right, side view of an endoscopic bipolar forceps showing a housing, a rotatable member, a shaft and an end-effector assembly in accordance with an embodiment of the present disclosure;

FIG. 2 is an enlarged, perspective view of an embodiment of the upper jaw assembly of the end-effector assembly of the forceps shown in FIG. 1 with parts separated in accordance with an embodiment of the present disclosure;

FIG. 3 is an enlarged, perspective view of an embodiment of the lower jaw assembly of the end-effector assembly of the forceps shown in FIG. 1 with parts separated in accordance with an embodiment of the present disclosure;

FIG. 4 is an enlarged, perspective view of an embodiment of one of the jaw members of an end-effector assembly, such as the jaw member of the lower jaw assembly shown in FIG. 3, with parts separated in accordance with the present disclosure;

FIG. 5 is an enlarged, perspective view of the assembled jaw member of FIG. 4 in accordance with an embodiment of the present disclosure;

FIG. 6 is an enlarged, perspective view of another embodiment of a jaw member, similar to the jaw member of the lower jaw assembly shown in FIG. 3, in accordance with the present disclosure;

FIG. 7 is an enlarged, perspective view of yet another embodiment of a jaw member, with parts separated, in accordance with the present disclosure;

FIG. 8 is a flowchart illustrating a method of manufacturing an end-effector assembly in accordance with an embodiment of the present disclosure;

FIG. 9 is a flowchart illustrating a method of manufacturing a jaw member of a jaw assembly in accordance with an embodiment of the present disclosure;

FIG. 10 is an enlarged, perspective view of a configuration of stop members on the inner-facing surface of a sealing plate, similar to the sealing plate of the jaw assembly shown in FIG. 2, in accordance with an embodiment of the present disclosure;

FIG. 11 is a an enlarged, perspective view of a configuration of rectangular-like recesses or channels defined in the sealing plate shown in FIG. 10 in accordance with an embodiment of the present disclosure;

FIG. 12 is a an enlarged, perspective view of a configuration of rectangular-like, texturized surface areas associated with the sealing plate shown in FIG. 10 in accordance with an embodiment of the present disclosure.

FIG. 13 is a an enlarged, perspective view of a configuration of stop members on the inner-facing surface of a sealing plate, similar to the sealing plate of the jaw assembly shown in FIG. 2, in accordance with another embodiment of the present disclosure;

FIG. 14 is a an enlarged, perspective view of a configuration of stop members on the inner-facing surface of a sealing plate, similar to the sealing plate of the jaw assembly shown in FIG. 2, in accordance with yet another embodiment of the present disclosure; and

FIG. 15 is a an enlarged, perspective view of a configuration of stop members on the inner-facing surface of a sealing plate, similar to the sealing plate of the jaw assembly shown in FIG. 2, in accordance with still another embodiment of the present disclosure.

### DETAILED DESCRIPTION

Hereinafter, embodiments of end-effector assemblies for use in electrosurgical instruments and methods of manufacturing jaw assembly components of end-effector assemblies of the present disclosure are described with reference to the accompanying drawings. Like reference numerals may refer to similar or identical elements throughout the description of the figures. As shown in the drawings and as used in this description, and as is traditional when referring to relative positioning on an object, the term “proximal” refers to that portion of the apparatus, or component thereof, closer to the user and the term “distal” refers to that portion of the apparatus, or component thereof, farther from the user.

This description may use the phrases “in an embodiment,” “in embodiments,” “in some embodiments,” or “in other embodiments,” which may each refer to one or more of the same or different embodiments in accordance with the present disclosure. For the purposes of this description, a phrase in the form “A/B” means A or B. For the purposes of the description, a phrase in the form “A and/or B” means “(A), (B), or (A and B)”. For the purposes of this description, a phrase in the form “at least one of A, B, or C” means “(A), (B), (C), (A and B), (A and C), (B and C), or (A, B and C)”.

Various embodiments of the present disclosure provide an electrosurgical forceps with an end-effector assembly including opposing jaw assemblies pivotably mounted with respect to one another. Various embodiments of the present disclosure provide jaw assemblies including jaw members formed to meet specific tolerance requirements for proper jaw alignment and other features, as by fineblanking. Various embodiments of the present disclosure provide methods of manufacturing jaw assembly components of end-effector assemblies for use in electrosurgical instruments, including without limitation, bipolar forceps.

Embodiments of the presently-disclosed electrosurgical forceps may be suitable for utilization in endoscopic surgical procedures and/or suitable for utilization in open surgical applications. Embodiments of the presently-disclosed bipolar forceps may be implemented using electromagnetic radiation at microwave frequencies, radio frequencies (RF) or at other frequencies. Electrosurgical systems including the presently-disclosed endoscopic bipolar forceps operatively coupled to an electrosurgical energy source according to various embodiments may be configured to operate at frequencies between about 300 KHz and about 10 GHz.

Various embodiments of the present disclosure provide an electrosurgical forceps with electrically non-conductive stop members associated with one or both of the opposing jaw assemblies. The presently-disclosed configurations of non-conductive stop members are designed to control the gap distance between opposing jaw assemblies, and may facilitate the gripping and manipulation of tissue during the sealing and dividing process.

Although the following description describes the use of an endoscopic bipolar forceps, the teachings of the present disclosure may also apply to a variety of electrosurgical devices that include end-effector assemblies assembled from tightly-toleranced jaw assembly components.

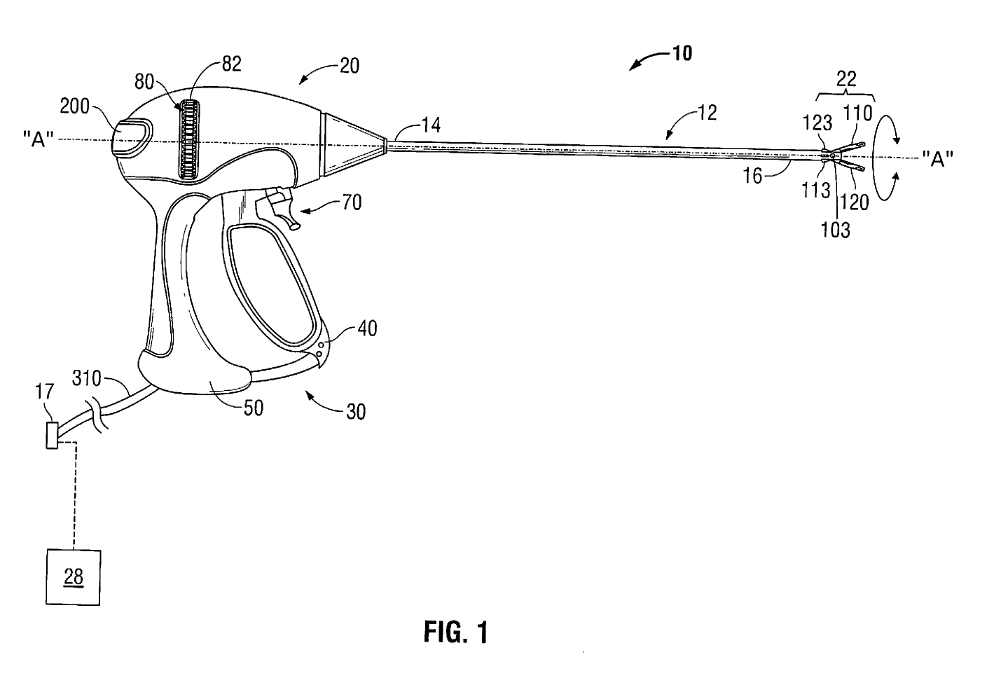

In FIG. 1, an embodiment of an endoscopic bipolar forceps 10 is shown for use with various surgical procedures and generally includes a housing 20, a handle assembly 30, a rotatable assembly 80, a trigger assembly 70 and an end-effector assembly 22 that mutually cooperate to grasp, seal and/or divide tubular vessels and vascular tissue (not shown). Although FIG. 1 depicts a bipolar forceps 10 for use in connection with endoscopic surgical procedures, the teachings of the present disclosure may also apply to more traditional open surgical procedures. For the purposes herein, the forceps 10 is described in terms of an endoscopic instrument; however, it is contemplated that an open version of the forceps may also include the same or similar operating components and features as described below.

Forceps 10 includes a shaft 12 that has a distal end 16 configured to mechanically engage the end-effector assembly 22 and a proximal end 14 configured to mechanically engage the housing 20. In some embodiments, the shaft 12 has a length from a proximal side of the handle assembly 30 to a distal side of the forceps 10 in a range of about 7 centimeters to about 44 centimeters. End-effector assembly 22 may be selectively and releaseably engageable with the distal end 16 of the shaft 12, and/or the proximal end 14 of the shaft 12 may be selectively and releaseably engageable with the housing 20 and the handle assembly 30.

The proximal end 14 of the shaft 12 is received within the housing 20, and connections relating thereto are disclosed in commonly assigned U.S. Pat. No. 7,150,097 entitled “METHOD OF MANUFACTURING JAW ASSEMBLY FOR VESSEL SEALER AND DIVIDER”, commonly assigned U.S. Pat. No. 7,156,846 entitled “VESSEL SEALER AND DIVIDER FOR USE WITH SMALL TROCARS AND CANNULAS”, commonly assigned U.S. Pat. No. 7,597,693 entitled “VESSEL SEALER AND DIVIDER FOR USE WITH SMALL TROCARS AND CANNULAS” and commonly assigned U.S. Pat. No. 7,771,425 entitled “VESSEL SEALER AND DIVIDER HAVING A VARIABLE JAW CLAMPING MECHANISM”.

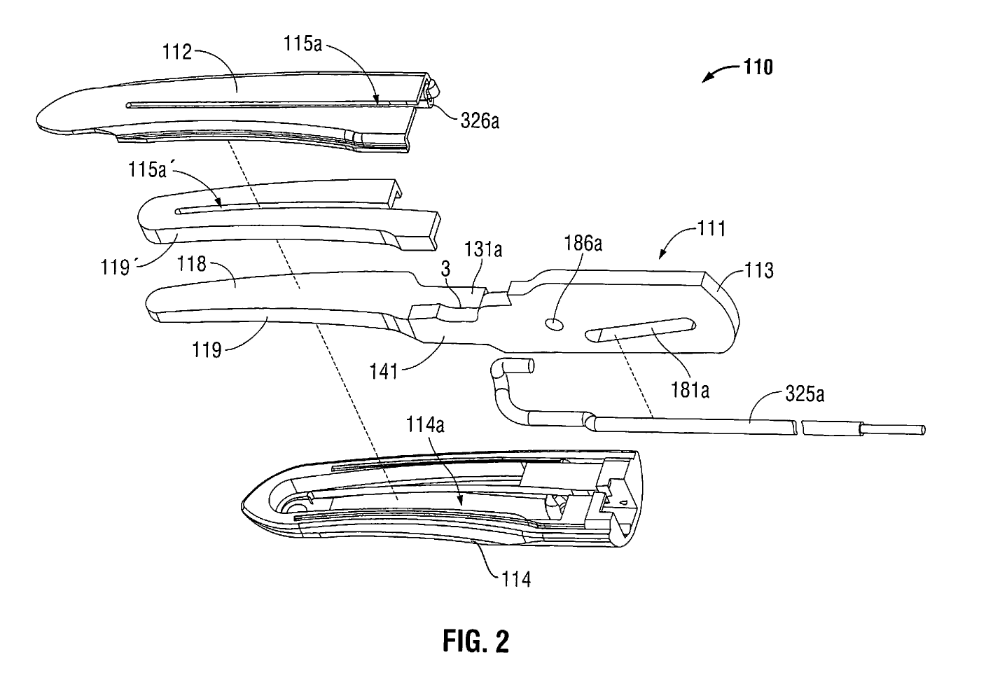

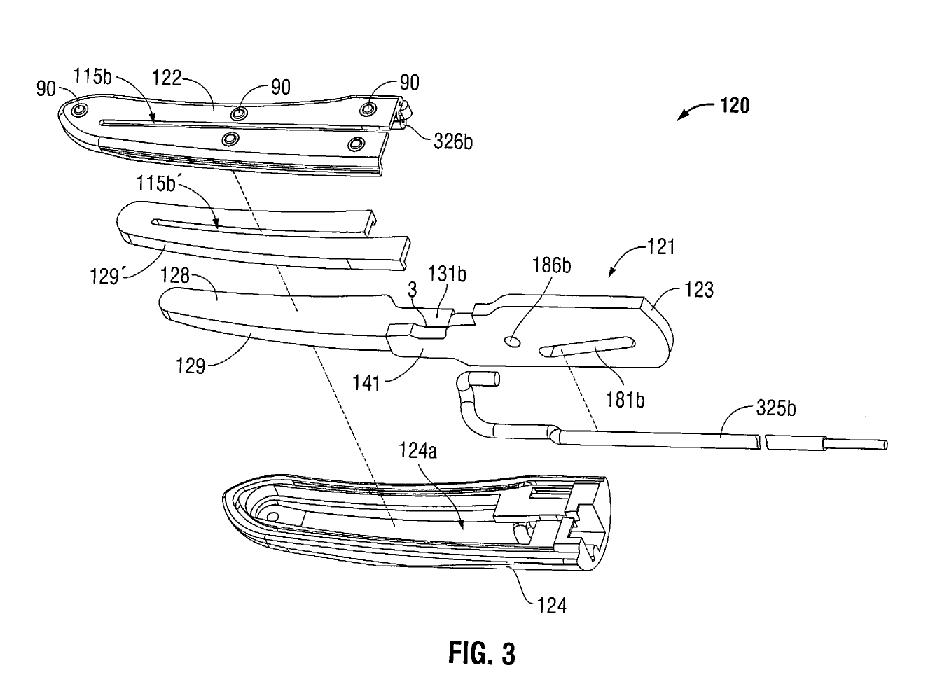

Forceps 10 includes an electrosurgical cable 310. Electrosurgical cable 310 may be formed from a suitable flexible, semi-rigid or rigid cable, and may connect directly to an electrosurgical power generating source 28. In some embodiments, the electrosurgical cable 310 connects the forceps 10 to a connector 17, which further operably connects the instrument 10 to the electrosurgical power generating source 28. Cable 310 may be internally divided into one or more cable leads (e.g., 325a and 325b shown in FIGS. 2 and 3, respectively) each of which transmits electrosurgical energy through their respective feed paths to the end-effector assembly 22.

Electrosurgical power generating source 28 may be any generator suitable for use with electrosurgical devices, and may be configured to provide various frequencies of electromagnetic energy. Examples of electrosurgical generators that may be suitable for use as a source of electrosurgical energy are commercially available under the trademarks FORCE EZ™, FORCE FX™, and FORCE TRIAD™ offered by Covidien. Forceps 10 may alternatively be configured as a wireless device or battery-powered.

End-effector assembly 22 generally includes a pair of opposing jaw assemblies 110 and 120 pivotably mounted with respect to one another. End-effector assembly 22 may be configured as a bilateral jaw assembly, i.e., both jaw assemblies 110 and 120 move relative to one another. Alternatively, the forceps 10 may include a unilateral assembly, i.e., the end-effector assembly 22 may include a stationary or fixed jaw assembly, e.g., 120, mounted in fixed relation to the shaft 12 and a pivoting jaw assembly, e.g., 110, mounted about a pivot pin 103 coupled to the stationary jaw assembly.

Jaw assembly 110 components including a jaw member 111 according an embodiment of the present disclosure are shown in FIG. 2. Jaw assembly 120 components including a jaw member 121 according to an embodiment of the present disclosure are shown in FIG. 3. Jaw assemblies 110 and 120 may include additional, fewer, or different components than shown in FIGS. 2 and 3, respectively, depending upon a particular purpose or to achieve a desired result. Jaw members 111 and 121, which are described in more detail later in this description, each include two or more components (e.g., fineblanked components), separately formed and subsequently joined together to form the respective jaw members 111 and 121. In some embodiments, the jaw member 111 includes a first arm member 113 (FIGS. 1 and 2) and a first support base 119 (FIG. 2), and the jaw member 121 includes a second arm member 123 (FIGS. 1, 3, 4 and 5) and a second support base 129 (FIGS. 3, 4 and 5).

As shown in FIG. 1, the end-effector assembly 22 is rotatable about a longitudinal axis “A-A” through rotation, either manually or otherwise, of the rotatable assembly 80. Rotatable assembly 80 generally includes two halves (not shown), which, when assembled about a tube of shaft 12, form a generally circular rotatable member 82. Rotatable assembly 80, or portions thereof, may be configured to house a drive assembly (not shown) and/or a knife assembly (not shown), or components thereof. A reciprocating sleeve (not shown) is slidingly disposed within the shaft 12 and remotely operable by the drive assembly (not shown). Examples of rotatable assembly embodiments, drive assembly embodiments, and knife assembly embodiments of the forceps 10 are described in the above-mentioned, commonly-assigned U.S. Pat. Nos. 7,150,097, 7,156,846, 7,597,693 and 7,771,425.

Handle assembly 30 includes a fixed handle 50 and a movable handle 40. In some embodiments, the fixed handle 50 is integrally associated with the housing 20, and the handle 40 is selectively movable relative to the fixed handle 50. Movable handle 40 of the handle assembly 30 is ultimately connected to the drive assembly (not shown). As can be appreciated, squeezing the movable handle 40 toward the fixed handle 50 pulls the drive sleeve (not shown) proximally to impart movement to the jaw assemblies 110 and 120 from an open position, wherein the jaw assemblies 110 and 120 are disposed in spaced relation relative to one another, to a clamping or closed position, wherein the jaw assemblies 110 and 120 cooperate to grasp tissue therebetween. Examples of handle assembly embodiments of the forceps 10 are described in the above-mentioned, commonly-assigned U.S. Pat. Nos. 7,150,097, 7,156,846, 7,597,693 and 7,771,425.

Forceps 10 includes a switch 200 configured to permit the user to selectively activate the forceps 10 in a variety of different orientations, i.e., multi-oriented activation. As can be appreciated, this simplifies activation. When the switch 200 is depressed, electrosurgical energy is transferred through one or more electrical leads (e.g., leads 325a and 325b shown in FIGS. 2 and 3, respectively) to the jaw assemblies 110 and 120. Switch 200 may be disposed on another part of the forceps 10 (e.g., the fixed handle 50, rotatable member 82, etc.) or another location on the housing assembly 20.

As best shown in FIG. 2, jaw member 111 includes a first support base 119 that extends distally from a first arm member 113. First arm member 113 and the first support base 119 are generally formed from metal, e.g., steel, and may include non-metal elements. First arm member 113 and the first support base 119 may be formed from any suitable material or combination of materials.

First arm member 113 and the first support base 119 are separately fabricated and each includes an engagement structure 141, 131a, respectively, configured for attachment to one another. During a manufacturing process, the engagement structure 141 of the first arm member 113 is welded, joined or otherwise attached to the engagement structure 131a of the first support base 119 to thereby form the jaw member 111 (hereinafter referred to as the “first jaw member”). As shown in FIGS. 3 through 5, engagement structures 141, 131b, which are described in more detail later in this description, are used in forming the jaw member 121 (hereinafter referred to as the “second jaw member”).

First arm member 113 may define one or more apertures at least partially therethrough, e.g., pivot holes and/or pin slots or openings. In some embodiments, as shown in FIG. 2, the first arm member 113 includes an elongated angled slot 181a and a pivot hole 186a defined therethrough. The shape, size and spacing of the slot 181a and the pivot hole 186a may be varied from the configuration depicted in FIG. 2. First arm member 113 may include additional, fewer, or different apertures than shown in FIG. 2. In some embodiments, a recess (e.g., recess 722 shown in FIG. 7) may be defined in the engagement structure of the first arm member (e.g., arm member 723 shown in FIG. 7).

In some embodiments, the support base 119 includes an inner-facing surface 118 configured to support an insulative substrate or insulator 119′ thereon. Insulator 119′, in turn, may be configured to support an electrically-conductive tissue-engaging surface or sealing plate 112 thereon. Sealing plate 112 may be affixed atop the insulator 119′ and support base 119 in any suitable manner, e.g., snap-fit, over-molding, stamping, ultrasonically welded, etc. Support base 119 together with the insulator 119′ may be encapsulated by the electrically-conductive tissue-engaging surface or sealing plate 112 and an outer housing 114. In some embodiments, the outer housing 114 is formed, at least in part, of an electrically non-conductive or substantially electrically non-conductive material.

Outer housing 114 includes a cavity 114a, e.g., configured to securely engage the electrically-conductive sealing plate 112. Cavity 114a may additionally, or alternatively, be configured to securely engage the support base 119 and the insulator 119′. This may be accomplished by stamping, by overmolding, by overmolding a stamped electrically-conductive sealing plate and/or by overmolding a metal injection-molded seal plate. Sealing plate 112 and the insulator 119′, when assembled, form a longitudinally-oriented slot or knife channel 115a, 115a′ defined therethrough for reciprocation of a knife blade (not shown). Insulator 119′ includes a channel 115a′ defined therein which extends along the insulating plate 119′ and which aligns in vertical registration with the knife channel 115a defined in the sealing plate 112 to facilitate translation of the distal end of the knife (not shown) therethrough. Examples of electrically-conductive sealing plate 112, outer housing 114, and knife blade embodiments are disclosed in commonly assigned International Application Serial No. PCT/US01/11412 filed on Apr. 6, 2001, entitled “ELECTROSURGICAL INSTRUMENT WHICH REDUCES COLLATERAL DAMAGE TO ADJACENT TISSUE”, and commonly assigned International Application Serial No. PCT/US01/11411 filed on Apr. 6, 2001, entitled “ELECTROSURGICAL INSTRUMENT REDUCING FLASHOVER”.

In some embodiments, jaw assembly 110 is connected to a first electrical lead 325a. Lead 325a, in turn, is electrically coupled with an electrosurgical energy source (e.g., 28 shown in FIG. 1). In some embodiments, lead 325a terminates within the outer insulator 114 and is configured to electro-mechanically couple to the sealing plate 112 by virtue of a crimp-like connection 326a.

As shown in FIG. 3, jaw assembly 120 includes similar elements to jaw assembly 110 of FIG. 2, such as an outer housing 124 having a cavity 124a defined therein and an insulative substrate or insulator 129′ configured to support an electrically-conductive tissue-engaging surface or sealing plate 122 thereon. Cavity 124a may be configured to at least partially encapsulate and/or securely engage the support base 129, the insulator 129′, and/or the electrically-conductive tissue-engaging surface or sealing plate 122. Second jaw member 121 includes a second support base 129 extending distally from a second arm member 123. Second arm member 123 and the second support base 129 may be formed from any suitable materials, e.g., metal, or combination of materials. Second arm member 123 and the second support base 129 are separately fabricated and each includes an engagement structure 141, 131b, respectively, configured for attachment to one another. During a manufacturing process, the engagement structure 141 of the second arm member 123 is welded, joined or otherwise attached to the engagement structure 131b of the second support base 129 to thereby form the second jaw member 121.

Second arm member 123 may define one or more apertures at least partially therethrough, e.g., pivot holes and/or pin slots or openings. In some embodiments, as shown in FIG. 3, the second arm member 123 includes an elongated angled slot 181a and a pivot hole 186a defined therethrough. In alternative embodiments not shown, the second arm member 123 may include other apertures defined at least partially therethrough.

Similar to like elements of jaw assembly 110, when assembled, the electrically-conductive tissue-engaging surface 122 and the insulator 129′, when assembled, include respective longitudinally-oriented knife channels 115b and 115b′ defined therethrough for reciprocation of a knife blade (not shown). When the jaw assemblies 110 and 120 are closed about tissue, knife channels 115a, 115a′ and 115b, 115b′ form a complete knife channel (not shown) to allow longitudinal extension of the knife blade (not shown) in a distal fashion to sever tissue along a tissue seal. In alternative embodiments, the knife channel may be completely disposed in one of the two jaw assemblies, e.g., jaw assembly 120, depending upon a particular purpose. Jaw assembly 120 may be assembled in a similar manner as described above with respect to jaw assembly 110.

As shown in FIG. 3, jaw assembly 120 is connected to an electrical lead 325b. Lead 325b, in turn, is electrically coupled to an electrosurgical energy source (e.g., 28 shown in FIG. 1). In some embodiments, lead 325b terminates within the outer insulator 124 and is configured to electro-mechanically couple to the sealing plate 122 by virtue of a crimp-like connection 326b. Leads 325a (FIG. 2) and 325b may allow a user to selectively supply either bipolar or monopolar electrosurgical energy to the jaw assemblies 110 and 120 as needed during surgery.

As best seen in FIG. 3, jaw assembly 120 includes a series of stop members 90 disposed on the inner-facing surface of the electrically-conductive tissue-engaging surface or sealing plate 122. Stop members 90 may be configured to facilitate and/or enhance the gripping and manipulation of tissue and to control the gap distance (not shown) between opposing jaw assemblies 110 and 120 during the sealing and cutting of tissue. Stop members 90 of varied configurations may be employed on one or both jaw assemblies 110 and 120 depending upon a particular purpose or to achieve a desired result. Examples of stop member embodiments as well as various manufacturing and assembling processes for attaching and/or affixing the stop members 90 to the electrically-conductive tissue-engaging surfaces 112, 122 are described in commonly-assigned International Application Serial No. PCT/US01/11413 filed on Apr. 6, 2001, entitled “VESSEL SEALER AND DIVIDER WITH NON-CONDUCTIVE STOP MEMBERS”. In some variations of stop members, compatible with any of the above embodiments, stop members may be printed, patterned, applied, or otherwise deposited using a direct write process, such as by a micro-capillary system, e.g., MICROPEN® technology, or any other suitable material deposition technology.

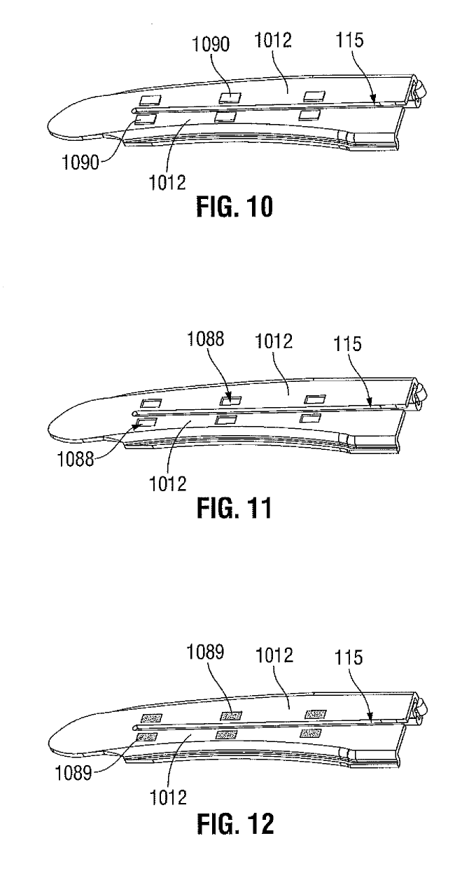

In some embodiments, as shown in FIGS. 10 through 15, described below, stop members (e.g., stop members 1090, 1390, 1490a, and 1590 shown in FIGS. 10, 13, 14 and 15, respectively) may be formed using a direct write process, e.g., MICROPEN® Technologies' MICROPENNING®, to deposit material atop the surface of an electrically-conductive tissue-engaging surface or sealing plate and/or into recesses or channels defined therein (e.g., channels 1088 shown in FIG. 11). In general, the term “direct write” describes a printing or patterning method that employs a computerized, motion-controlled stage with a motionless pattern generating device to dispense flowable materials in a designed pattern onto a surface.

MICROPENNING® is a micro-capillary technology that uses a positive displacement method of pumping flowable materials, typically having a viscosity of between about 5 and about 500,000 centipoise, onto a surface. In some embodiments, using MICROPENNING® direct writing to precisely control the volume of flowable material (e.g. dielectric ink, or other suitable material) applied, in one or more layers, to an electrically-conductive tissue-engaging surface or sealing plate, results in the formation of stop members that meet specific tolerance requirements for controlling the gap distance between opposing jaw assemblies 110 and 120.

FIG. 10 shows a first series of rectangular-like stop members 1090 disposed on an electrically-conductive tissue-engaging surface or sealing plate 1012 on one side of a knife channel 115 and a second series of rectangular-like stop members 1090 disposed on the sealing plate 1012 on the other side of the knife channel 115. Sealing plate 1012 shown in FIGS. 10 through 12 is similar to the sealing plate 112 of FIG. 2, and further description thereof is omitted in the interests of brevity.

Stop members 1090 may be formed, in one or more layers, of any suitable dielectric material, e.g., a dielectric ink. The first series of stop members 1090 and/or the second series of stop members 1090 may be formed using a direct write process, e.g., MICROPEN® Technologies' MICROPENNING®, or other suitable material deposition technology. In some embodiments, as shown in FIG. 10, stop members 1090 are substantially equal in size; however, one or more of the stop members 1090 may be dimensioned larger or smaller than the other stop members 1090 depending upon a particular purpose or to achieve a desired result.

FIG. 11 shows a configuration of rectangular-like recesses or channels 1088 defined in the electrically-conductive tissue-engaging surface or sealing plate 1012 of FIG. 12 on both sides of the longitudinally-oriented knife channel 115. Channels 1088 may be configured to receive a volume of dielectric ink, prior to or during the formation of stop members 1090 shown in FIG. 10, e.g., to enhance robustness of stop members 1090, minimize the negative effects of ink spread, and/or prevent or reduce undesired rounding of the intended rectangular-like perimeter of the stop members 1090 due to ink spreading and/or surface tension. Channels 1088 may be formed in the sealing plate 1012 by any suitable process, such as precision stamping, injection molding, etc. The shape and size of the channels 1088 may be varied from the configuration depicted in FIG. 11.

It is to be understood that the configuration of stop members 1090 shown in FIG. 10 (and channels 1088 shown in FIG. 11) are merely illustrative and non-limiting examples of stop members (and channels for use in forming stop members) associated with an electrically-conductive tissue-engaging surface or seal plate 1012, and that jaw assembly embodiments of the present disclosure may utilize many different configurations of stop members, some with fewer, or additional, stop members than depicted in FIG. 10. In alternative embodiments not shown, the channels 1088 defined in the sealing plate 1012 may include additional features, such as protrusions or stepped edges, e.g., to facilitate secure attachment of stop members 1090 to the sealing plate 1012.

FIG. 12 shows a plurality of rectangular-like, texturized surface areas 1089 disposed on, or otherwise associated with, the electrically-conductive tissue-engaging surface or sealing plate 1012 on both sides of the longitudinally-oriented knife channel 115. The texturized surface areas 1089 may include any suitable type of texturized surface or pattern formed by any suitable process. In some embodiments, as shown in FIG. 12, the texturized surface areas 1089 may be similar in some aspects, e.g., in shape and relative position, to the configuration of rectangular-like stop members 1090 shown in FIG. 10, e.g., to facilitate the positioning and/or secure attachment of stop members 1090 to the sealing plate 1012.

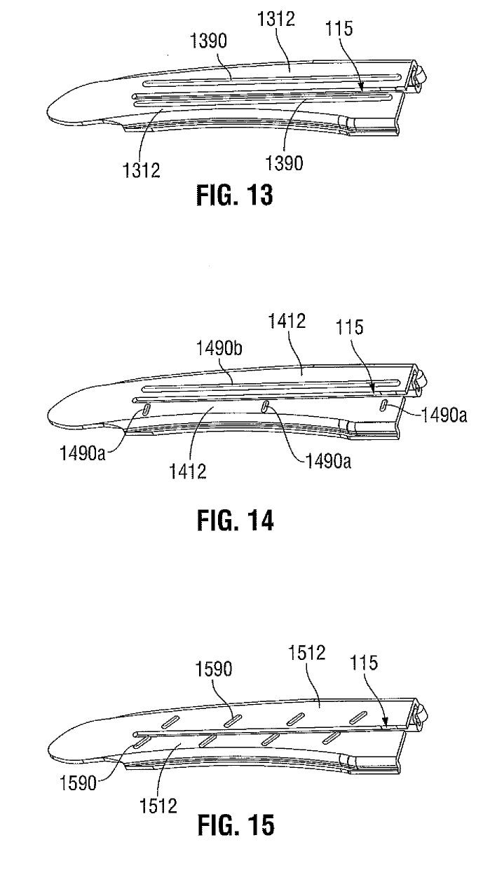

In some embodiments, as shown in FIG. 13, an electrically-conductive tissue-engaging surface or sealing plate 1312 includes two stop members 1390 configured as longitudinally-oriented projections or ridges disposed on the inner-facing surface of an electrically-conductive tissue-engaging surface or sealing plate 1312 on both sides of a knife channel 115. Sealing plate 1312 shown in FIG. 13 is similar to the sealing plate 112 of FIG. 2, and further description thereof is omitted in the interests of brevity.

In alternative embodiments not shown, one or more stop members 1390 may be disposed on either opposing jaw assembly (e.g., opposing jaw assemblies 110 and 120 shown in FIG. 1) on opposite sides of the longitudinally-oriented knife channel and/or in an alternating, laterally-offset manner relative to one another along the length of the surface of either or both jaw members. Stop members 1390 may be formed using a direct write process, e.g., MICROPEN® Technologies' MICROPENNING®.

FIG. 14 shows a stop member 1490b configured as a longitudinally-oriented ridge disposed atop a sealing plate 1412 on one side of a knife channel 115 and a configuration of stop members 1490a disposed on the other side of the knife channel 115. Sealing plate 1412 shown in FIG. 14 is similar to the sealing plate 112 of FIG. 2, and further description thereof is omitted in the interests of brevity. Stop members 1490a and/or stop member 1490b may be formed using a direct write process, e.g., MICROPENNING®.

FIG. 15 shows another configuration of stop members 1590 disposed on, or otherwise associated with, the electrically-conductive tissue-engaging surface or sealing plate 1512 on both sides of a longitudinally-oriented knife channel 115. Sealing plate 1512 shown in FIG. 15 is similar to the sealing plate 112 of FIG. 2, and further description thereof is omitted in the interests of brevity. Stop members 1590 may be formed using a direct write process, e.g., MICROPENNING®.

The presently-disclosed jaw members (e.g., first jaw member 111 and second jaw member 121 shown in FIGS. 1 and 2, respectively) may be implemented as a modularized component assembly. The engagement structures 141 and 131a, 131b may be used for joining together jaw arm members and support bases of varied geometries, e.g., lengths and curvatures, or having additional, fewer, or different features than the first and second support bases 119, 129 and the first and second arm members 113, 123, such that the variously-configured arm members and support bases may be separately fabricated and assembled into jaw member configurations suitable for various end-effector configurations, e.g., depending upon design of specialized electrosurgical instruments.

In alternative embodiments not shown, compatible with any of the above embodiments of arm members and support bases for assembly into jaw member configurations, an electrically-insulative hinge may be used to electrically isolate the opposing jaw members from one another, wherein a configuration of stop members may be disposed on the inner-facing surface of the support base of either or both jaw members. In either or both jaw members, the support base may include a configuration of recesses or channels, e.g., formed by fineblanking, for use in forming stop members, e.g., to facilitate the positioning and/or secure attachment of stop members to the support base.

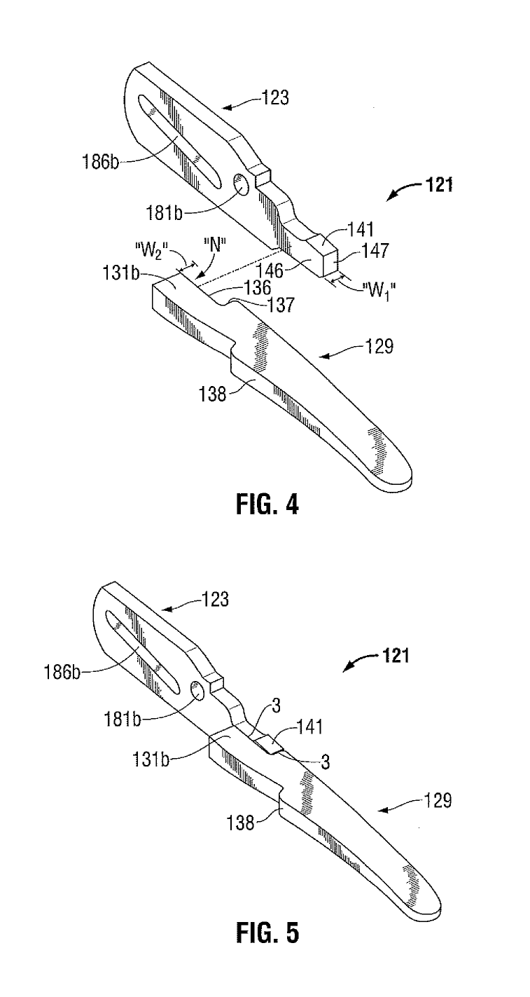

FIGS. 4 and 5 show an embodiment the second jaw member 121 of the jaw assembly 120 shown in FIG. 3. Second jaw member 121 and/or the first jaw member 111 (FIG. 2) may be manufactured using fineblanking. The precision of fineblanking, which is described in more detail later in this description, generally means that the presently-disclosed jaw members may be formed with closely-toleranced features, including without limitation, pivot holes, pin slots, openings, and/or various engagement-structure features, e.g., to achieve proper jaw alignment and a high level of structural integrity.

In FIG. 5, the assembled jaw member 121 is shown and includes the second support base 129 extending distally from the second arm member 123, wherein the engagement structure 131b of the second support base 129 is joined to the engagement structure 141 of the second arm member 123 along an interface 3 therebetween. Engagement structure 131b of the second support base 129 and the engagement structure 141 of the second arm member 123 may be joined by a welding-type process, e.g., laser welded, or joined together by other suitable process, e.g., adhesively joined. As shown in FIG. 4, the second support base 129 includes a body portion 138 that extends distally from the engagement structure 131b. A cut-out or notch “N” is cooperatively defined by the body portion 138 and the engagement structure 131b. In some embodiments, the notch “N” may have a width “W₂” substantially equal to the width “W₁” of the engagement structure 141 of the second arm member 123, e.g., to generally align outer lateral surfaces of the arm member 123 and the support base 129.

As shown in FIGS. 4 and 5, notch “N” is configured to receive therein the engagement structure 141 of the second arm member 123 when the opposing inner lateral surfaces 146 and 136 of the engagement structures 141 and 131b, respectively, are brought together. As shown in FIGS. 4 and 5, when assembled, a distal end 147 of the engagement structure 141 of the second arm member 123 is disposed substantially adjacent to a proximal end 137 of the body portion 138.

In alternative embodiments not shown, the inner lateral surface 146 of the engagement structure 141 of the second arm member 123 (and/or first arm member 113) and/or the inner lateral surface 136 of the engagement structure 131b of the second support base 129 (and/or first support base 119) may include detents, tongue and groove interfaces, locking tabs, adhesive ports, etc., utilized either alone or in combination for assembly purposes.

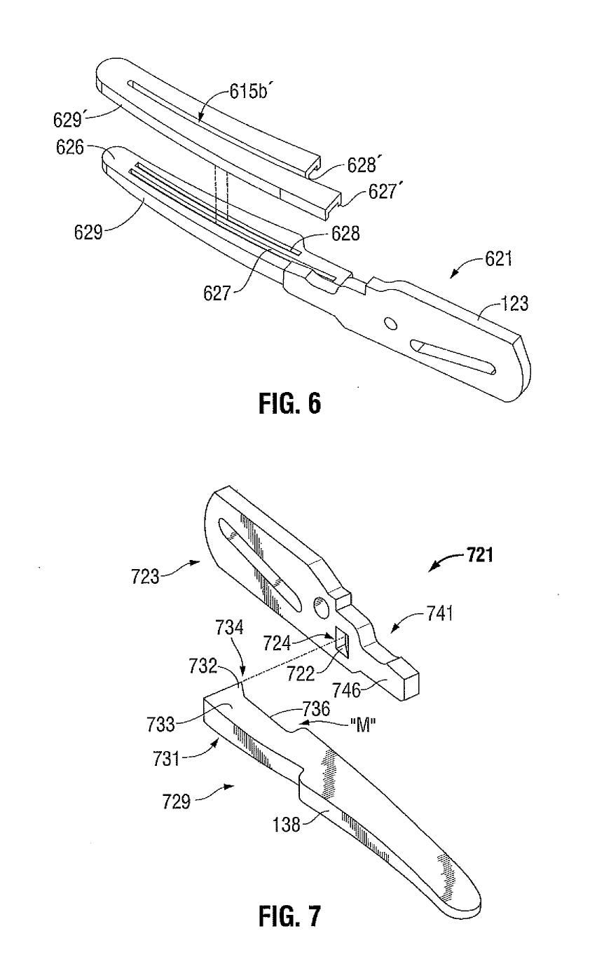

FIG. 6 shows an embodiment of a jaw member 621 and an insulator 629′ of a jaw assembly, such as the jaw assembly 120 shown in FIG. 3. In some embodiments, the insulator 629′ may be formed by a molding process, e.g., injection molding, or other suitable manufacturing process, and may be formed of any suitable electrically non-conductive material. Insulator 629′ is similar to the insulator 129′ shown in FIG. 3, except for the first and second flanges 62T and 628′, respectively, disposed along the knife channel 615b′ defined in the insulator 629′ of FIG. 6, and further description of the like features is omitted in the interests of brevity.

Jaw member 621 shown in FIG. 6 is similar to the second jaw member 121 shown in FIG. 3, except for the first and second longitudinally-extending grooves or channels 627 and 628, respectively, formed into the upper surface 626 of the support base 629 of the jaw member 621 of FIG. 6, and further description of the like elements is omitted in the interests of brevity. First and second channels 627, 628, which may be formed by fineblanking, are configured to receive the first and second flanges 627′, 628′ therein. In some embodiments, an adhesive layer (not shown) may be provided to the first channel 627, or portion thereof, and/or the second channel 628, or portion thereof, e.g., to adhesively bond the first flange 627′, or portion thereof, and/or the second flange 628′, or portion thereof, to the support base 629 of the jaw member 621 via the first channel 627 and/or the second channel 628. First and second flanges 627′, 628′ of the insulator 629′ may be affixed within the first and second channels 627, 628 of the support base 629 in any suitable manner, e.g., snap-fit, ultrasonically welded, etc.

Jaw member 621 and the insulator 629′, when assembled, including the first and second flanges 627′ and 628′ received in the respective first and second channels 627 and 628, may increase stability of the knife channel and/or provide increased jaw member integrity, and/or may facilitate and/or improve knife-blade reciprocation, and/or may result in improved tissue-cutting capabilities, as compared to the jaw assembly embodiments shown in FIGS. 2 and 3.

FIG. 7 shows an embodiment of a jaw member 721 including an arm member 723 and a support base 729. Jaw member 721 shown in FIG. 7 is similar to the second jaw member 121 shown in FIG. 4, except for the configuration of the engagement structure 731 of the support base 729 and the configuration of the engagement structure 741 of the arm member 723 of FIG. 7, and further description of the like elements is omitted in the interests of brevity.

Support base 729 shown in FIG. 7 includes an engagement structure 731 and the body portion 138 that extends distally from the engagement structure 731. A cut-out or notch “M” is cooperatively defined by the body portion 138 and the engagement structure 731. Notch “M” is configured to receive therein the engagement structure 741 of the arm member 723 when the opposing inner lateral surfaces 746 and 736 of the engagement structures 741 and 731, respectively, are brought together. Notch “M” is similar to the notch “N” shown in FIG. 4, and further description thereof is omitted in the interests of brevity.

Engagement structure 731 of the support base 729 includes a first alignment member 734 configured to engage with a second alignment member 724 defined in the engagement structure 741 of the arm member 723. In some embodiments, the second alignment member 724 includes a recess 722 defined in the inner lateral surface 746 of the engagement structure 741, and the first alignment member 734 includes a protrusion 732 extending outwardly of the inner lateral surface 736 of the engagement structure 731 configured to engage (e.g., matingly engage) with the recess 722. Recess 722 defined in the engagement structure 741 of the arm member 723 is configured to receive the protrusion 732 of the alignment member 734 therein. In some embodiments, as shown in FIG. 7, the alignment member 724 is disposed at the proximal end of the engagement structure 731. The shape, size and location of the first alignment member 734 and the second alignment member 724 may be varied from the configuration depicted in FIG. 7.

During assembly of the jaw member 721, the alignment members 724, 734 may facilitate and/or improve alignment of the arm member 723 and the support base 729. When the engagement structures 741, 731 are brought together and joined, the respective alignment members 724, 734 may increase the structural integrity of the jaw member 721, when assembled, as compared to the jaw members of the jaw assembly embodiments shown in FIGS. 2 and 3.

Various embodiments of the present disclosure provide jaw members including arm members and support bases, each including engagement structures configured for attachment to one another, which may be formed using fineblanking processes to achieve specific tolerance requirements for proper jaw alignment and other closely-toleranced features. Jaw member 121, or component(s) thereof (e.g., arm member 123 and/or support base 129), jaw member 111, or component(s) thereof (e.g., arm member 113 and/or support base 119), jaw member 621, or component(s) thereof (e.g., arm member 123 and/or support base 629) and/or jaw member 721, or component(s) thereof (e.g., arm member 723 and/or support base 729) may be formed using fineblanking.

Fineblanking, a hybrid metal-forming process combining the technologies of metal stamping and cold extrusion, may be used to achieve flatness and cut edge characteristics that may be unobtainable by conventional stamping and punching methods. During conventional punching, when a punch makes contact with the sheet of metal stock, the metal tends to deform and bulge around the point of the initial punch contact. Using conventional methods that allow the metal to bulge or plastically deform during the cutting process results in straining of the metal, which, in turn, causes stress. Trapped stresses in a part may cause it to lose its flatness.

In general, fineblanking operations require the use of high-pressure pads and are carried out on triple-action hydraulic presses on which the punch, guide plate, and die movements can be controlled individually or simultaneously. The pads hold the metal flat during the cutting process and prevent the metal from plastically deforming during punch entry. Fineblanking can be used on a variety of metals, including stainless steels. Fineblanked parts are usually made with rolled stock, which makes the parts inherently stronger than powder metal compositions and cast components. Using fineblanking, a part's cut surface is sheared smoothly over the entire workpiece thickness, with minimal die roll on edges. Achievable part dimensional tolerances may range from about +/−0.0003 inches to about +/−0.002 inches, depending upon material thickness, material characteristics (e.g., tensile strength), and part layout.

The use of fineblanking processes allows excellent dimensional control, accuracy and repeatability throughout a production run. The stability and precision of fineblanking processes generally means that operations such as grinding, milling, forming, shaving, and leveling can be eliminated. Fineblanking can be used to produce, in a single step, a part that would require multiple operations, set-ups, and man-hours using other processes. Fineblanking may be more economical for large production runs than conventional operations when additional machining cost and time are factored.

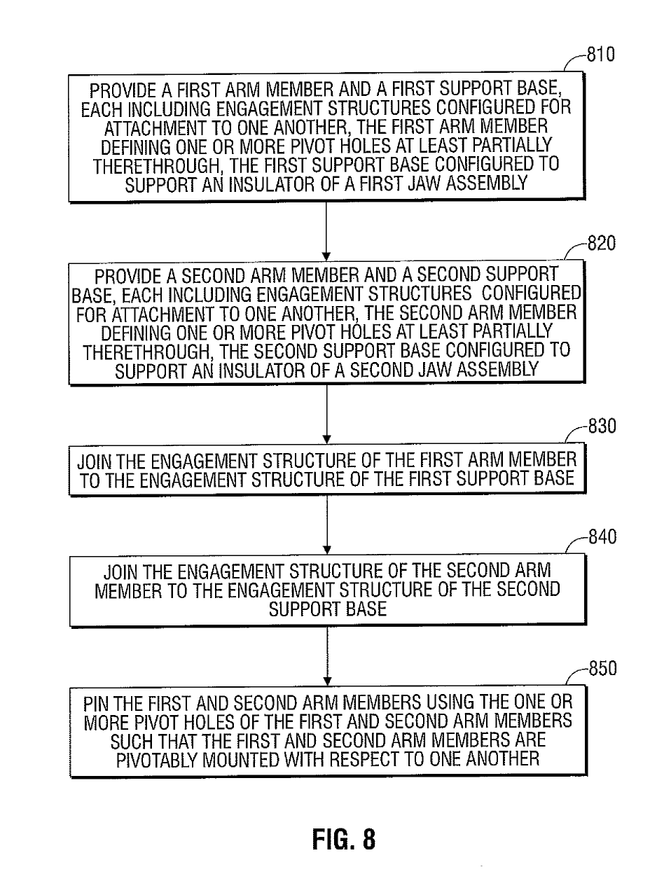

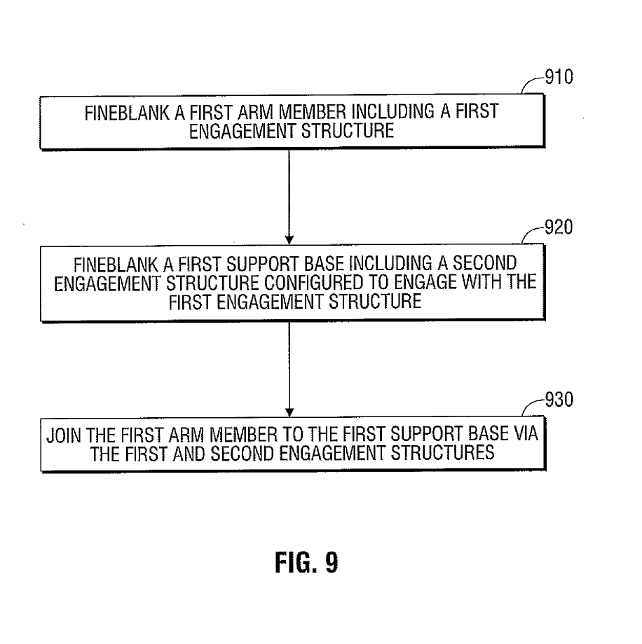

Hereinafter, a method of manufacturing an end-effector assembly is described with reference to FIG. 8 and a method of manufacturing a jaw member of a jaw assembly is described with reference to FIG. 9. It is to be understood that the steps of the methods provided herein may be performed in combination and in a different order than presented herein without departing from the scope of the disclosure.

FIG. 8 is a flowchart illustrating a method of manufacturing an end-effector assembly 22 according to an embodiment of the present disclosure that includes a first jaw assembly 110 and a second jaw assembly 120. In some embodiments, the first and second jaw members 110, 120 are configured to be pivotably mounted with respect to one another.

In step 810, a first arm member 113 and a first support base 119 are provided, including engagement structures 141, 131a, respectively, that are configured for attachment to one another. First arm member 113 and/or the first support base 119 may be formed using a fineblanking process. First arm member 113 defines at least partially therethrough one or more apertures, e.g., pivot holes, and/or pin slots or openings. In some embodiments, the first arm member 113 includes an elongated angled slot 181a and a pivot hole 186a defined therethrough. In some embodiments, the first support base 119 includes an inner-facing surface 118 configured to support an insulative substrate or insulator 119′ associated with the first jaw assembly 110.

In step 820, a second arm member 123 and a second support base 129 are provided, each including engagement structures 141, 131b, respectively, cooperatively configured for attachment to one another. Second arm member 123 and/or the second support base 129 may be formed using a fineblanking process. First arm member 123 defines at least partially therethrough one or more apertures, e.g., pivot holes, and/or pin slots or openings. In some embodiments, the first arm member 113 includes an elongated angled slot 181b and a pivot hole 186b defined therethrough.

In some embodiments, the second support base 129 includes a body portion 138 that extends distally from the engagement structure 131b of the second support base 129, wherein the engagement structure 131b of the second support base 129 and the body portion 138 cooperatively define a notch “N” configured to receive therein the engagement structure 141 of the second arm member 123. In some embodiments, the body portion 138 is configured to support an insulative substrate or insulator 129′ associated with the second jaw assembly 120.

In step 630, the engagement structure 141 of the first arm member 113 is joined to the engagement structure 131a of the first support base 119 to thereby form a first jaw member 111 of a first jaw assembly 110. In some embodiments, the engagement structure 141 of the first arm member 113 is joined to the engagement structure 131a of the first support base 119 along an interface 3 formed therebetween when the engagement structure 141 is placed in contact with the engagement structure 131a. Engagement structure 141 of the first arm member 113 and the engagement structure 131a of the first support base 119 may be joined by a welding-type process, e.g., laser welded, or joined together by other suitable process.

In step 840, the engagement structure 141 of the second arm member 123 is joined, e.g., welded, to the engagement structure 131b of the second support base 129 to thereby form a second jaw member 121 of a second jaw assembly 120.

In step 850, the first and second arm members 113, 123 (and/or jaw members 111, 121) are pinned using the one or more pivot holes 186a, 186b of the first and second arm members 113, 123 such that the first and second arm members 113, 123 (and/or jaw assemblies 110, 120) are pivotably mounted with respect to one another. Pinning the first and second arm members 113, 123 (and/or jaw members 113, 123), in the step 850, may include the steps of providing a pivot pin 103 and inserting the pivot pin 103 through the one or more pivot holes 186a, 186b of the first and second arm members 113, 123 such that the jaw assemblies 110 and 120 are capable of pivoting about the pivot pin 103. It will be appreciated that additional manufacturing steps may be undertaken after the step 840, prior to pinning of the first and second jaw members 113, 123 in the step 850.

FIG. 9 is a flowchart illustrating a method of manufacturing a jaw member of a jaw assembly in accordance with an embodiment of the present disclosure. In step 910, fineblanking is used to form a first arm member 123 including a first engagement structure 141. First arm member 123 may define one or more apertures at least partially therethrough, e.g., pivot holes and/or pin slots or openings. In some embodiments, first arm member 123 includes an elongated angled slot 181b and a pivot hole 186b defined therethrough.

In step 920, fineblanking is used to form a first support base 129 including a second engagement structure 131b. Second engagement structure 131b is configured to engage with the first engagement structure 141. In some embodiments, the first support base 129 includes an inner-facing surface 128 configured to support an insulative substrate or insulator 129′ associated with a first jaw assembly 120.

In step 930, the first arm member 123 is joined to the first support base via the first and second engagement structures 141, 131b. Joining the first arm member 123 to the first support base 129 via the first and second engagement structures 141, 131b, in step 930, may include the step of welding the first engagement structure 141 to the second engagement structure 131b, e.g., along an interface 3 formed therebetween when opposing inner surfaces 146, 136 of the first and second engagement structures 141, 131b are in intimate contact with one another.

In alternative embodiments, compatible with any of the above embodiments, a jaw assembly may include one or more stop members formed using a direct write process to deposit a dielectric ink on an inner-facing surface of an electrically-conductive tissue-engaging surface associated with the first support base.

The above-described bipolar forceps is capable of directing energy into tissue, and may be suitable for use in a variety of procedures and operations. The above-described end-effector embodiments may utilize both mechanical clamping action and electrical energy to effect hemostasis by heating tissue and blood vessels to coagulate, cauterize, cut and/or seal tissue. The jaw assemblies may be either unilateral or bilateral. The above-described bipolar forceps embodiments may be suitable for utilization with endoscopic surgical procedures and/or hand-assisted, endoscopic and laparoscopic surgical procedures. The above-described bipolar forceps embodiments may be suitable for utilization in open surgical applications.

The above-described end-effector embodiments may include one or more non-conductive stop members associated with one or both of the opposing jaw assemblies. A direct write process, e.g., MICROPENNING®, may be used to deposit a dielectric ink on the inner-facing surface of an electrically-conductive tissue-engaging surface associated with one or both of the opposing jaw assemblies.

The above-described method of manufacturing an end-effector assembly and method of manufacturing a jaw member of a jaw assembly may result in the formation of jaw assemblies that meet specific tolerance requirements for proper jaw alignment and other tightly-toleranced jaw assembly features. The above-described jaw members include separately-formed arm members and support bases, which may be formed using fineblanking processes, each including engagement structures configured for attachment to one another. The above-described arm members and support bases formed by fineblanking may include various closely-toleranced features, e.g., pivot holes, pin slots, openings, grooves or channels and/or various engagement-structure features, which may facilitate proper jaw alignment and a high level of structural integrity in the manufacture of jaw assemblies.

Although embodiments have been described in detail with reference to the accompanying drawings for the purpose of illustration and description, it is to be understood that the inventive processes and apparatus are not to be construed as limited thereby. It will be apparent to those of ordinary skill in the art that various modifications to the foregoing embodiments may be made without departing from the scope of the disclosure.

What is claimed is:

1. An end-effector assembly, comprising:
opposing first and second jaw assemblies pivotably mounted with respect to one another, wherein the first jaw assembly includes a first jaw member and the second jaw assembly includes a second jaw member;
the first jaw member including:
a first arm member defining at least one aperture at least partially therethrough; and
a first support base extending distally from the first arm member, wherein an engagement structure of the first arm member is joined to an engagement structure of the first support base to thereby form the first jaw member;
the second jaw member including:
a second arm member defining at least one aperture at least partially therethrough; and
a second support base extending distally from the second arm member, wherein an engagement structure of the second arm member is joined to an engagement structure of the second support base to thereby form the second jaw member; and
at least one pivot pin engaged with the at least one apertures of the first and second jaw members such that the first and second jaw assemblies are pivotably mounted with respect to one another.

2. The end-effector assembly of claim 1, wherein the first support base is configured to support an insulator associated with the first jaw assembly.

3. The end-effector assembly of claim 1, wherein the first support base includes a body portion that extends distally from the engagement structure of the first support base, wherein the engagement structure of the first support base and the body portion cooperatively define a notch configured to receive therein the engagement structure of the first arm member.

4. The end-effector assembly of claim 3, wherein the body portion is configured to support an insulator associated with the first jaw assembly.

5. The end-effector assembly of claim 4, wherein the first jaw assembly further includes a sealing surface affixed atop the insulator.

6. The end-effector assembly of claim 5, wherein the first jaw assembly is adapted to connect the sealing surface associated therewith to an electrosurgical generator.

7. The end-effector assembly of claim 1, wherein the second support base is configured to support an insulator associated with the second jaw assembly.

8. The end-effector assembly of claim 1, wherein the second support base includes a body portion that extends distally from the engagement structure of the second support base, the engagement structure of the second support base and the body portion cooperatively defining a notch configured to receive therein the engagement structure of the second arm member.

9. The end-effector assembly of claim 8, wherein the body portion is configured to support an insulator associated with the second jaw assembly.

10. The end-effector assembly of claim 9, wherein the second jaw assembly further includes a sealing surface affixed atop the insulator.

11. The end-effector assembly of claim 10, wherein the second jaw assembly is adapted to connect the sealing surface associated therewith to an electrosurgical generator.

12. A method of manufacturing an end-effector assembly, comprising the steps of:
providing a first arm member and a first support base, each including engagement structures configured for attachment to one another, the first arm member defining at least one pivot hole at least partially therethrough;
providing a second arm member and a second support base, each including engagement structures configured for attachment to one another, the second arm member defining at least one pivot hole at least partially therethrough;
joining the engagement structure of the first arm member to the engagement structure of the first support base;
joining the engagement structure of the second arm member to the engagement structure of the second support base; and
pinning the first and second arm members using the at least one pivot hole of the first and second arm members such that the first and second arm members are pivotably mounted with respect to one another.

13. The method of manufacturing an end-effector assembly of claim 12, wherein at least one of the first arm member and the first support base are formed using a fineblanking process.

14. The end-effector assembly of claim 12, wherein the first support base includes a body portion that extends distally from the engagement structure of the first support base, the engagement structure of the first support base and the body portion cooperatively defining a notch configured to receive therein the engagement structure of the first arm member.

15. The end-effector assembly of claim 12, wherein the second support base includes a body portion that extends distally from the engagement structure of the second support base, the engagement structure of the second support base and the body portion cooperatively defining a notch configured to receive therein the engagement structure of the second arm member.

16. The method of manufacturing an end-effector assembly of claim 15, wherein the body portion is configured to support an insulator associated with the second jaw assembly.

17. The method of manufacturing an end-effector assembly of claim 12, further comprising the steps of:
providing an electrically-conductive tissue-engaging surface associated with the first support base; and
forming at least one stop member using a direct write process to deposit a dielectric ink on an inner-facing surface of the electrically-conductive tissue-engaging surface.

18. The method of manufacturing an end-effector assembly of claim 12, wherein the step of pinning the first and second jaw members using the at least one pivot hole of the first and second arm members includes the steps of:
providing a pivot pin; and
inserting the pivot pin through the at least one pivot hole of the first and second arm members such that the first and second jaw assemblies are capable of pivoting about the pivot pin.

19. A method of manufacturing a jaw member, comprising the steps of:
fineblanking a first arm member including a first engagement structure;
fineblanking a first support base including a second engagement structure configured to engage with the first engagement structure; and
joining the first arm member to the first support base via the first and second engagement structures.

20. The method of manufacturing a jaw member of claim 19, wherein the step of joining the first arm member to the first support base via the first and second engagement structures includes the step of welding the first engagement structure to the second engagement structure along an interface formed therebetween when the first and second engagement structures are in intimate contact with one another.
